(19) United States
(12) Patent Application Publication
(10) Pub. No.: US 2023/0285151 A1
(43) Pub. Date: Sep. 14, 2023

(54) A METHOD OF MAKING A MODIFIED POLYMER-BASED INDIVIDUAL 3D PRINTED BIORESORBABLE BONE IMPLANT FOR USE IN TRAUMATOLOGY AND ORTHOPEDICS

(71) Applicant: Advanced Development of Additive Manufacturing, Inc., Stamford, CT (US)

(72) Inventors: Arkadii Bohdan, Lymanka Village (UA); Vadym Volkov, Lymanka Village (UA); Oleg Rogankov, Odessa (UA)

(21) Appl. No.: 18/041,335

(22) PCT Filed: Aug. 10, 2021
(86) PCT No.: PCT/US2021/045384

§ 371 Date: Feb. 10, 2023

Related U.S. Application Data
(60) Provisional application No. 63/063,483, filed on Aug. 10, 2020.
(60) Provisional application No. 63/063,485, filed on Aug. 10, 2020.

(51) Int. Cl. A61F 2/28; B29C 64/118; B29C 64/393; B29C 64/209; A61L 27/46

(52) U.S. Cl. CPC A61F 2/28; A61L 27/46; B29C 64/118; B29C 64/209; B29C 64/393; A61F 2002/30062

(57) ABSTRACT

The present disclosure relates to bio-resorbable bone implants made from a material composition made from a polymer and calcium-based minerals. The proposed composition is suitable for 30 printing. The bone implant of the present invention was created using a 30 extrusion-based printing technology. The method of making the implant and the composition of the inventive implant were optimized to enable improved osteoconductive activity at the transplantation site. The novel composition and process enables the replacement of the implant with native bone tissue, which is expected during the treatment process.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

This application is a National Stage application under 35 U.S.C. §371 of International Application No. PCT/US2021/045384, having an International Filing Date of Aug. 10, 2021, which, claims priority to U.S. Provisional Application No. 63/063,483, filed on Aug. 10, 2020, and U.S. Provisional Application No. 63/063/485, filed on Aug. 10, 2020, all of which are considered part of the disclosure of this application, and are incorporated in their entireties herein.

### FIELD OF THE INVENTION

Tissue engineering is an interdisciplinary field that applies the principles of engineering, medicine, basic sciences to develop tissue substitutes - implants that restore, maintain or improve the function of human tissues. Large-scale cultivation of human, animal or artificial origin cells can provide materials to replace damaged components in humans.

Natural or synthetic materials when implanted into the human body as temporary structures provide a framework that allows the body’s own cells to migrate, differentiate, grow and form new tissues, while the framework itself is gradually absorbed by the body.

The requirements for a polymer implant must meet the following criteria: it must be an open interconnective network to stimulate cell growth and transfer of nutrients and metabolic waste. The biocompatibility and bioresorbability with controlled rates of decomposition and resorption have to match the rate of tissue replacement in the body. There must be a suitable surface composition for cell attachment, proliferation and differentiation. And it must offer high mechanical properties corresponding to the tissues at the site of implantation.

Furthermore, the structure of the implant must protect the interior of the proliferating cells of the pore network and their extracellular matrix from mechanical overload for a sufficient period of time. This is especially important for supporting tissues such as bones and cartilage.

### BACKGROUND

Layer-by-layer fusion deposition modeling (FDM) is a well-known additive manufacturing process that forms three-dimensional objects by extrusion and deposition of individual layers of thermoplastic materials.

Generally, before any printing, one would first start with a generated model of the implant on a computer. The model is imported into specialized software which enables the use of this model for 3D printing.

The FDM method involves extrusion of a melted material through a heated nozzle, followed by a deposition in the form of thin solid layers on the substrate. The thermoplastic polymer material is fed to a temperature-controlled FDM extrusion printing head, where it is heated to a semi-fluid state. The printing head spreads the material through a nozzle in ultra-thin layers with high accuracy.

After printing the material returns from melted to solid state. Layer-by-layer fabrication allows one to design porous, interconnected structures for general applications in medicine, and the pore morphology can vary according to the implant structure.

BJP, binder-jet printing, is also a well-known method for printing polymer-based materials. Here, BJP is not suitable due to the fact that it uses powder bases and a liquid binder to bind particles.

In the case of polycaprolactone, which is usually used in granular form, it will be difficult to use it as a powder, since it will not sufficiently bind with water- or glycerin-based binders during the printing stage. It would present a further challenge in the post-processing of the product using high-temperature sintering, as is the traditional approach in the binder-jet printing, due to the fact that the chemical composition of polycaprolactone degrades at temperatures higher than 300° C.

Hydroxyapatite is an artificial bioresorbable material which is neutral for the human body. It is well-known to use hydroxyapatite to make implants. Hydroxyapatite is the chemical compound of Calcium and Phosphorus with chemical formula Ca₁₀ (PO₄)₆ (OH)₂ and particle size up to 100 nanometers.

Hydroxyapatite particles are utilized to stimulate the vascularization and differentiation of macrophages and mesenchymal stem cells to osteogenic cells. In addition, the main products of hydroxyapatite resorption help to buffer the by-products of the acidic resorption of aliphatic polyester and thereby help avoid the formation of an unfavorable environment for the cells due to the low pH.

In addition, polycaprolactone (PCL) is a semi-crystalline, bioresorbable polymer belonging to the family of aliphatic polyesters. It has favorable properties for thermoplastic processing.

It has a low glass transition temperature of -60° C., a melting point of 60° C., and a high decomposition temperature of 350° C., with a wide range of temperatures that allow extrusion. PCL is currently considered to be a biodegradable material compatible with soft and hard human tissue.

FDM printing technology allows us to use a modified polymer in 3D printing to give it the desired individual shape with high accuracy, printing speed and the ability to control the printing process. Also, the FDM technology allows us to optimize the range of temperatures to use the inventive polymers without the danger of damaging their chemical structure, which can lead to the loss of the desired properties of the implant.

### BRIEF DESCRIPTION OF THE DRAWINGS

The present embodiments are illustrated by way of example and are not intended to be limited by the figures of the accompanying drawings.

FIG. 1 is an example of a 3d-printed implant showing a cross-section of a patella or a kneecap according to the present invention.

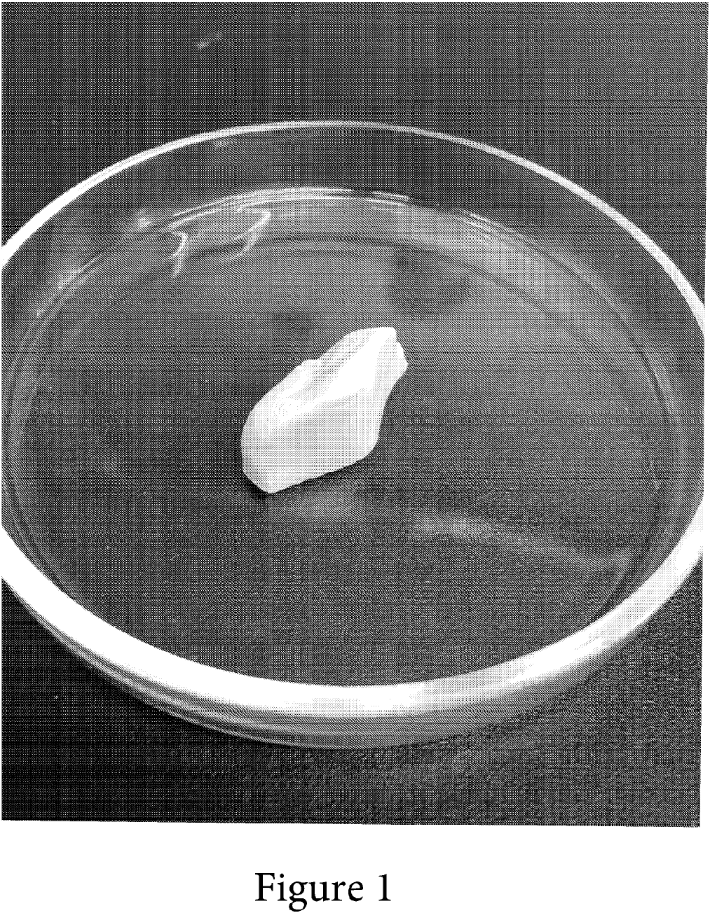

FIG. 2 shows representative designs of the patella implant cross-section according to the present invention.

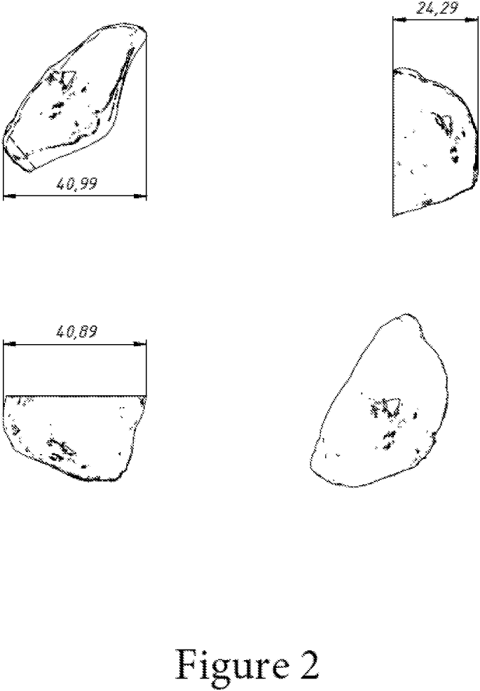

FIG. 3 is an example of a 3d-printed polymer-based sample with an internal lattice structure exhibiting a microporosity pattern with a distance between the lattices of 500 microns.

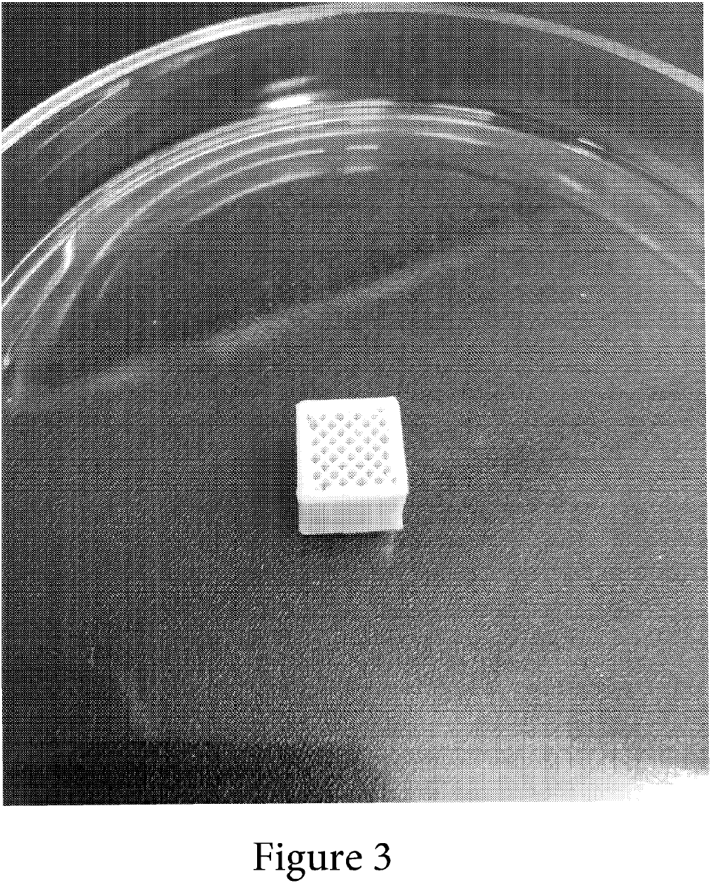

FIG. 4 is an example representation of a bone, an implant and a composition in accordance with the present invention.

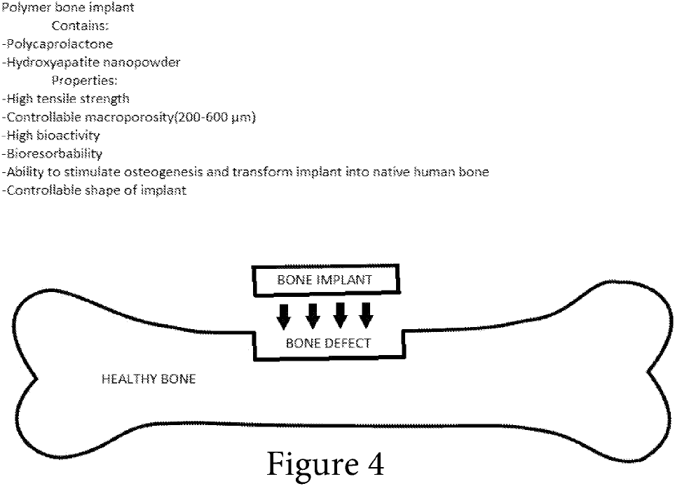

### BRIEF DESCRIPTION OF THE INVENTION

Since polycaprolactone and hydroxyapatite are chosen for the implant composition, this leads to limitations in the choice of printing technology. Generally speaking, the FDM process offers the following benefits: it enables the use of a wide range of biomaterials; it enables the ability to make bio-composites with the necessary mechanical and biological properties; it enables the flexibility to make objects of the desired shape, which can be specified in the 3D model; it offers a quick and affordable way to print products; the resultant implant meets the necessary compliance with aseptic conditions, and the subsequent sterilization of products allows their use for medical purposes.

### DETAILED DESCRIPTION

The present invention method utilizes a FDM printing method to process a bioresorbable composite of two biomaterials to make a novel bone implant, wherein a biodegradable polymer (polycaprolactone, PCL) is mixed with a calcium-based powder (hydroxyapatite, HA) to make a compound that meets all the criteria for use in general tissue engineering applications.

Furthermore, we propose a design of the implant which mimics the structure of a tubular bone with a solid outer layer and a lattice inside to imitate a spongy bone. The inner part of the implant is made in the form of a lattice with a bar diameter of 200-500 µm and a spacing of 200-500 µm between the rods.

This structure increases the surface area of the implant, creates controlled macroporosity to stimulate angiogenesis and it attracts the mesenchymal differentiation of stem cells into osteogenic and bone tissue cells at the site of the implant.

The three-dimensional polymer matrix of the implant of the present invention has the kinetics of degradation and resorption from 6 to 18 months and the ability to maintain a given shape under the action of the biomechanical load.

The inclusion of calcium-based material in the bioresorbable polymer provides support for the composite material that improves the rates of degradation and resorption. This composite material improves the biocompatibility and integration of solid tissues.

Hydroxyapatite particles that are embedded in the matrix of a synthetic polymer provide increased osteogenic differentiation and connective tissue growth compared to the more hydrophobic surface of the polymer. In addition, the main products of hydroxyapatite resorption help to buffer the by-products of the acidic resorption of aliphatic polyester and thereby help avoid the formation of an unfavorable environment for the cells of hard tissues due to the low pH.

Since the polycaprolactone-hydroxyapatite composition itself is not porous, the inventive process enables a novel lattice structure inside the implant to provide the implant body with conditions for the growth of soft tissues into the thickness of the implant and a better process of implant engraftment and its subsequent resorption.

### Exemplary Composition Overview

TABLE 1

Exemplary embodiment with percentages of the composition and its benefits

| Component | Range | Optimal percentage | Benefit of optimal percentage |
|---|---|---|---|
| hydroxyapatite | 0-40% | 20% | Enables optimal cell activity on the implant surface |
| polycaprolactone | 60-100% | 80% | Provides a biodegradable matrix for implant stability and high density parameters |

### Exemplary Process Parameters Overview

TABLE 2

Exemplary embodiment with optimal processing parameters

| Parameter | Range | Optimal Value | Benefits & goals |
|---|---|---|---|
| Mixture melting (T°C) | 100-300 | 190 | Complete mixture homogenization (which is critical for printing quality and biological properties) |
| Extrusion nozzle size (mm) | up to 3 | 1.75 | Enables for obtaining a thread from the melted mixture |
| Printer head extruder (T°C) | 0-300 | 160 | Complete melting of the thread for further forming of an implant |
| Printer bed (T°C) | 0-100 | 0 | Enables better adhesion of a lower layer of an implant with the printer bed surface |
| Printing speed (mm/s) | 1-200 | 25 | Controls the movement speed of a printer head which forms an implant on the printer bed |
| Thread soaking time (h) | 0-72 | 24 | Required for antiseptic measures and for chemical excluding of the monomer parts from the polymer composition that could remain after melting process. |
| Implant soaking time (h) | 0-72 | 24 | Required for antiseptic measures and for chemical excluding of the monomer parts from the polymer composition that could remain after melting process. |
| Ethanol solution percentage (%) | 1-99 | 70 | Required for antiseptic measures and for chemical excluding of the monomer parts from the polymer composition that could remain after melting process. |

The mixture melting is preferably set to 190° C., because this is optimal temperature of melting of PCL pellets. When the mixture melting temperature is <100° C., it is inefficient because the mixture will not completely melt to retain the desired properties of the final implant. When the mixture melting temperature is >300° C., it is inefficient because the chemical composition will be damaged.

The extrusion machine nozzle and printer head nozzle can be optimized to fit the printer and standards. Most commonly used diameters are 1.63 mm, 1.75 mm and 3 mm.

The printer head extruder T°C is preferably set to 160° C. for optimal extrusion to limit or prevent the damage to the polymer chemical composition.

The printer bed temperature is preferably set to 0° C. At >100° C., the conditions are inefficient, because the implant can be deformed by constant heating effect on the lower layers of the implant. The composition and process conditions which result in the chemical properties of the implant are optimized to prevent the implant deformation effect.

The printing speed is preferably set to 25 mm/s to provide accuracy in the printing process. At a printing speed of more than 50 mm/s, the process will cause poor-quality printing. It may result in smears of the composition and defects during printing due to fast movement of the printer head. When the printer head moves too fast, it does not allow to accurately apply the extrudable composition to the specified coordinates.

The thread soaking time and implant soaking time are preferably set to 24 hours. When the soaking times exceed 72 hours, the process is inefficient because the number of microorganisms and particles that form on the surface of the material.

### Exemplary Method 1

One embodiment of the system of the present invention is comprised of the following devices:
Compounding machine
Thread extruder
FDM 3D-printer

One embodiment of the process of the present invention is comprised of the following steps:
1. First, to make the compound for extrusion, mixing hydroxyapatite nanopowder up to 40% and polycaprolactone pellets up to 90% to create a homogeneous mixture. Forming a thread for extrusion by melting the homogenous mixture at 180-200° C., followed by an extrusion in an extrusion machine with the nozzle up to 3 mm.
2. Then, utilizing a 3D implant printing process, wherein simulating a 3D model in Autodesk 3DS Max using the results of a patient’s CT/MRI examination. Followed by a setup of the 3D printer in the following set parameters: extruder T = 160° C., bed T = 0° C., printing speed = 25 mm/s, nozzle diameter = 1.75 mm. Soaking the thread in 70% ethanol for up to 72 hours to make a filament; Loading the filament into a 3D printer; Printing the implant; Soaking the printed implant in 70% ethanol for up to 72 hours.
3. Sterilization of the implant for sterile packaging.
4. Surgical implantation to the site of a defect according to the traumatic methods of surgical interventions.

### Example Embodiments

1. A method for producing a modified polymer-based bioresorbable implant comprising: making a compound of a calcium-based powder up to 40% and a biodegradable polymer up to 90% to create a homogeneous mixture;
creating a thread by melting obtained homogeneous mixture at up to 250° C. and extrusion in an extrusion machine with the nozzle up to 3 mm;
soaking the thread in the antiseptic solution for up to 72 hours;
creating a 3D-model of implant for printing;
adjusting the 3D printer to the set parameters, wherein the extruder T is adjusted up to 200° C., the bed default setting is set to 0, which can be generally equivalent to a room T or 25° C., the printing speed is adjusted up to 100 mm/s, and the printing head nozzle is adjusted up to 3 mm; printing the 3D-model of the implant using a FDM 3D-printer to make a printed implant;
soaking the implant in the antiseptic solution for up to 72 hours;
drying the implant for up to 24 hours.

2. The method according to claim 1, wherein hydroxyapatite nanopowder is used as the calcium-based powder.

3. The method according to claim 1, wherein polycaprolactone pellets are used as the biodegradable polymer.

4. The method according to claim 1, wherein the composition of a homogeneous mixture is 10% of hydroxyapatite nanopowder and 90% of polycaprolactone pellets.

5. The method according to claim 1, wherein the composition of a homogeneous mixture is 20% of hydroxyapatite nanopowder and 80% of polycaprolactone pellets.

6. The method according to claim 1, wherein the composition of a homogeneous mixture is 30% of hydroxyapatite nanopowder and 70% of polycaprolactone pellets.

7. The method according to claim 1, wherein the ethanol solution is used as an antiseptic solution for soaking the thread.

8. The method according to claim 7, wherein the concentration of an ethanol solution is up to 80%.

9. The method according to claim 1, wherein the thread soaking time is set to 24 hours.

10. The method according to claim 1, wherein the 3D printer parameters are set to: extruder temperature is 160° C., bed temperature is 0° C., printing speed is 25 mm/s.

11. The method according to claim 1, wherein the ethanol solution is used as an antiseptic solution for soaking the implant.

12. The method according to claim 11, wherein the concentration of an ethanol solution is up to 80%.

13. The method according to claim 1, wherein the implant soaking time is set to 24 hours.

14. The method according to claim 1, wherein the calcium-based powder is selected from the group of chemical compounds consisting of calcium and/or phosphorus with the Ca/P ratio of 1.5-1.67, tricalcium phosphate, monocalcium phosphate, dicalcium phosphate, tetracalcium phosphate, hydroxyapatite, alpha-tricalcium phosphate, beta-tricalcium phosphate, calcium oxide(II) and mixtures thereof.

15. The method according to claim 1, wherein the extrusion machine nozzle diameter is 1.75 mm.

16. The method according to claim 1, wherein the extrusion machine nozzle diameter is 3 mm.

17. The method according to claim 1, wherein the printing head nozzle diameter is 1.75 mm.

18. The method according to claim 1, wherein the printing head nozzle diameter is 3 mm.

What is claimed is:

1. A method for producing a modified polymer-based bioresorbable implant comprising:
making a compound of up to 40% a calcium-based powder and up to 90% a biodegradable polymer to create a homogeneous mixture;
creating a thread by melting the homogeneous mixture at a temperature of up to 250° C. and extruding in an extrusion machine with a nozzle diameter up to 3 mm: soaking the thread in an antiseptic solution for up to 72 hours;
creating a 3D-model of an implant for printing;
adjusting a 3D printer to a plurality of set parameters, wherein an extruder temperature is adjusted up to 200° C., a bed default setting is set to 0, wherein 0 is equivalent to 25° C., a printing speed is adjusted up to 100 mm/s, and a printing head nozzle is adjusted up to 3 mm;
printing a 3D-model of the implant using a FDM 3D-printer to make a printed implant;
soaking the implant in the antiseptic solution for up to 72 hours; and
drying the implant for up to 24 hours.

2. The method of claim 1, wherein the calcium-based powder comprises hydroxyapatite nanopowder.

3. The method of claim 1, wherein the biodegradable polymer comprises polycaprolactone pellets.

4. The method of claim 1, wherein the a homogeneous mixture comprises 10% hydroxyapatite nanopowder and 90% polycaprolactone pellets.

5. The method of claim 1, wherein the homogeneous mixture comprises 20% hydroxyapatite nanopowder and 80% polycaprolactone pellets.

6. The method of claim 1, wherein the a homogeneous mixture comprises 30% hydroxyapatite nanopowder and 70% polycaprolactone pellets.

7. The method of claim 1, wherein the antiseptic solution for soaking the thread comprises an ethanol solution.

8. The method of claim 7, wherein the concentration of the ethanol solution is up to 80%.

9. The method of claim 1, wherein the thread soaking time is set to 24 hours.

10. The method of claim 1, wherein the 3D printer parameters comprise: extruder temperature is 160° C., bed temperature is 0° C., printing speed is 25 mm/s.

11. The method of claim 1, wherein the antiseptic solution for soaking the implant comprises an ethanol solution.

12. The method of claim 11, wherein the concentration of the ethanol solution is up to 80%.

13. The method of claim 1, wherein the implant soaking time is set to 24 hours.

14. The method of claim 1, wherein the calcium-based powder is selected from the group of chemical compounds consisting of calcium and/or phosphorus with the Ca/P ratio of 1.5-1.67, tricalcium phosphate, monocalcium phosphate, dicalcium phosphate, tetracalcium phosphate, hydroxyapatite, alpha-tricalcium phosphate, beta-tricalcium phosphate, calcium oxide(II), and mixtures thereof.

15. The method of claim 1, wherein the extrusion machine nozzle diameter is 1.75 mm.

16. The method of claim 1, wherein the extrusion machine nozzle diameter is 3 mm.

17. The method of claim 1, wherein the printing head nozzle diameter is 1.75 mm.

18. The method of claim 1, wherein the printing head nozzle diameter is 3 mm.
